(19) United States
(12) Patent Application Publication
(10) Pub. No.: US 2015/0018973 A1
(43) Pub. Date: Jan. 15, 2015

(54) Feet Extensions

(71) Applicant: Prince Rattan Rana, Normal, IL (US)

(72) Inventor: Prince Rattan Rana, Normal, IL (US)

(21) Appl. No.: 13/851,916

(22) Filed: Jul. 12, 2013

(51) Int. Cl. A61F 2/66

(52) U.S. Cl. CPC A61F 2/66; A61F 2002/6614; A61F 2002/5003
USPC 623/29

(57) ABSTRACT

Feet Extensions are improved version of custom height shoes for leg syndrome. Leg length discrepancy & Short stature. Feet Extensions are worn over the feet to add height in short leg. Unlike traditional shoes, feet extensions can be worn in shower, bathtub, and swimming pool. User can sleep in bed with feet extensions on their feet. Feet Extensions are made of silicone rubber in skin tone. Feet Extensions look more natural shape matching user feet.

Feet Extensions—A device worn over the feet to add height in short leg or both legs comprising: one piece flexible silicone/rubber hollow inside; thick at the bottom; Generally human feet shaped with five open holes in front hollow inside; hole at top for feet to slide in; in human skin color tone; raised circular set of lines (2 small in front bottom part and 1 big bottom at heel) for traction for wet locations.

Invention is used for: Feet Extensions are used to add height in short leg syndrome, Leg length discrepancy & Short stature.

How it works: User slide feet inside Feet extension like traditional shoe. User wears them over the feet, adding height in one leg or both legs. User can wear traditional shoes over the feet extensions as feet extensions are very thin yet strong and flexible.

### CROSS-REFERENCE TO RELATED APPLICATIONS

This application is a continuation of U.S. patent application No. 13851916, filed 27 Mar. 2013

### STATEMENT REGARDING FEDERALLY SPONSORED RESEARCH (IF APPLICABLE)

Not Applicable

### BACKGROUND OF THE INVENTION

My wife has one leg that is shorter than the other leg (cerebral palsy). And although there are shoes that are different heights, it is not recommended to wear them 27/7. For example it is difficult to wear them each time she gets up in the middle of the night to go to the bathroom, in the shower/bath, swimming pools, etc.

Feet Extensions are made of silicone and look like feet. This gives the opportunity to add a few inches under the feet. The design has open toes and are open at the top of the natural foot to help circulate air. The silicone is soft material and will help to absorb the shock when walking. This will decrease damage to the leg joints.

Feet Extensions can be worn at all times without the need to take them off for shower/bath or bed. It can be worn instead of shoes and will work like part of body. Feet Extensions will be made the color of skin to make them look like part of body.

### SUMMARY OF THE INVENTION

Problem: A person who has one leg that is shorter than the other wobbles a lot when they walk (my wife has one leg that is shorter than the other leg, her condition is cerebral palsy). And although, there are shoes that have different heights to help with the short leg. Those shoes aren't recommended to wear all the time. They can't be worn in the bath/shower, to bed, at the pool, etc. Not to mention they are bulky, heavy and don't look good.

Solution: Feet extensions look like part of the feet made in silicone in skin color, person can wear them like socks or shoes and it will add few inches of height in short leg, making it easier to walk around in the home, kitchen, shower/bath etc. Users can even sleep wearing their feet extensions improving their quality of life and saving them the hassle of wearing traditional height shoes in home.

### DESCRIPTION OF THE DRAWINGS

Please see image for reference.

FIG. 1 is a isometric view of feet extension.

FIG. 2 is a right side isometric view of feet extension,

FIG. 3 is a bottom View, Raised circular lines to Improves traction.

FIG. 4 is a backside view of feet extension.

FIG. 5 is a front view of feet extension.

FIG. 6 is a side view of feet extension.

FIG. 7 is a half cut view from left side.

FIG. 8 is a left side view of feet extension on user's foot

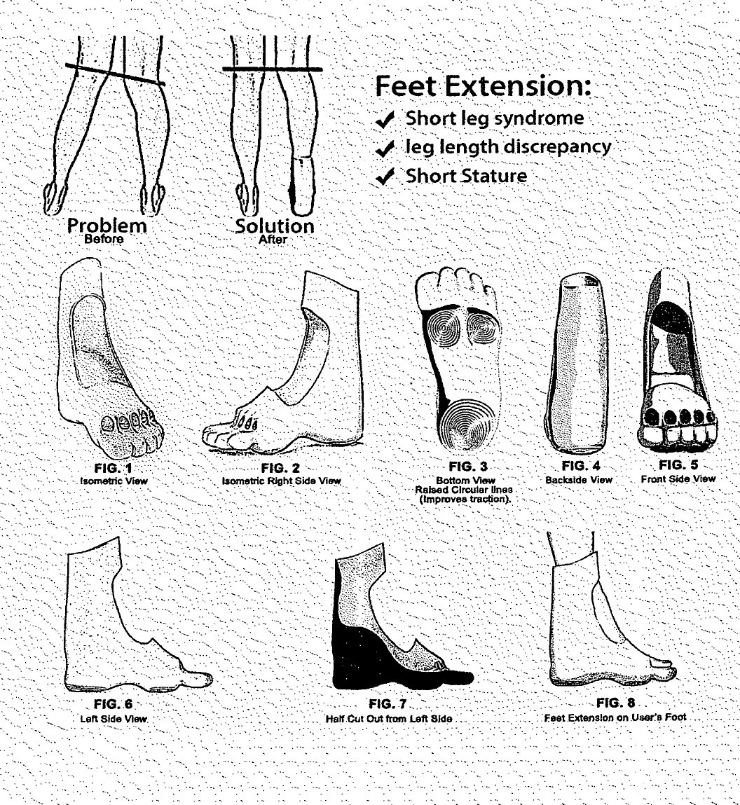

### DETAILED DESCRIPTION OF THE INVENTION

Feet Extensions: An improved version of Height shoes for people suffering from short leg syndrome, Leg length discrepancy & short stature. Feet extensions will be manufactured with 3d printers in skin color, with silicone material. The material will be flexible enough to adjust to the users feet. It will have open toes, which will provide great grip on users feet and will keep most of the feet exposed for air circulation, and open toes will make feet extensions stay in its place. It will be flexible to fit a majority of the users costing them $100 per feet extension and $200 for a pair.

Feet extensions will improve the life of millions of people around the world suffering from leg syndrome, Leg length discrepancy & Short stature. Feet Extensions can be made in different colors matching user skin tone to make it more natural on the user's feet.

### Advantages of the Invention Compared to Shoes

Feet extensions have several advantages over traditional shoes:

They can be worn . . .
In bed, no need to take them off before you go to bed, they are made of soft (silicone) material will not hurt you or your loved ones in bed.
In shower, open toes design provides strong grip and at bottom raised circular lines for traction (see no illustration no. 11) in wet locations.
In bath tub.
In Swimming pools, user can wear feet extensions in swimming pools (see no illustration no. 11)
In wet locations

How to use Feet Extensions:

Use both hands to hold feet extensions and slide feet inside feet extensions.

I claim:

1. Feet Extensions
A device worn over the feet to add height in short leg/legs
A device worn over the feet to add height in short leg or both legs comprising: one piece flexible silicone/rubber hollow inside; thick at the bottom; Generally human feet shaped with five open holes in front hollow inside; hole at top for feet to slide in; in human skin color tone; raised circular set of lines (2 small in front bottom part and 1 big bottom at heel) for traction for wet locations.
In means for adding height to feet in wet locations such shower, bathtubs, swimming pools. Or
In means for adding additional height to for short leg syndrome, leg length discrepancy & short stature in humans
